(19) United States
(12) Patent Application Publication
(10) Pub. No.: US 2025/0160676 A1
(43) Pub. Date: May 22, 2025

(54) AMBIENT SNORE DETECTION ON IOT DEVICES WITH MICROPHONES

(71) Applicant: Samsung Electronics Co., Ltd., Suwon-si (KR)

(72) Inventors: Wei Sun, Allen, TX (US); Hao-Hsuan Chang, Plano, TX (US); Se Ho Kim, Richardson, TX (US); Hao Chen, Allen, TX (US); Doyoon Kim, Seoul (KR); Taeyong Song, Seoul (KR); Joonhyun Lee, Seoul (KR)

(21) Appl. No.: 18/909,770

(22) Filed: Oct. 8, 2024

Related U.S. Application Data
(60) Provisional application No. 63/599,968, filed on Nov. 16, 2023.

(51) Int. Cl. A61B 5/08; A61B 5/00

(52) U.S. Cl. CPC A61B 5/0816; A61B 5/4818; A61B 2562/0204

(57) ABSTRACT

An electronic device includes a processor and a microphone. The microphone is configured to send audio, from an ambient environment of the electronic device, to the processor. The processor is configured to process, based on a current step size of an audio stream segmenter, the audio received from the microphone into an audio segment. The processor is further configured to determine whether the audio segment includes a snoring sound, and set a next step size of the audio stream segmenter based on the determination whether the audio segment includes the snoring sound.

### CROSS-REFERENCE TO RELATED APPLICATION AND CLAIM OF PRIORITY

This application claims priority under 35 U.S.C. § 119(e) to U.S. Provisional Patent Application No. 63/599,968 filed on Nov. 16, 2023. The above-identified provisional patent application is hereby incorporated by reference in its entirety.

### TECHNICAL FIELD

This disclosure relates generally to electronic devices. More specifically, this disclosure relates to ambient detection on IoT devices with microphones.

### BACKGROUND

The ability to detect snoring has several applications and use cases. For example, snoring event monitoring can be used to assist sleep studies since snoring happens most frequently in the light sleep stage. Detecting snoring can also be utilized in the early detection of obstructive sleep apnea (OSA), which is one of the most common sleep disorders that can increase risks of hypertension, cardiovascular disease, and stroke. Conventional methods for snoring detection are usually expensive due to the high cost of a dedicated machine and the labor fee of a medical technician for operating the machine.

### SUMMARY

This disclosure provides methods and apparatuses for ambient snore detection on IoT devices with microphones.

In one embodiment, an electronic device is provided. The electronic device includes a processor and a microphone. The microphone is configured to send audio, from an ambient environment of the electronic device, to the processor. The processor is configured to process, based on a current step size of an audio stream segmenter, the audio received from the microphone into an audio segment. The processor is further configured to determine whether the audio segment includes a snoring sound, and set a next step size of the audio stream segmenter based on the determination whether the audio segment includes the snoring sound.

In another embodiment, a method of operating an electronic device is provided. The method includes processing, based on a current step size of an audio stream segmenter, audio from an ambient environment of the electronic device received from a microphone, into an audio segment. The method also includes determining whether the audio segment includes a snoring sound, and setting a next step size of the audio stream segmenter based on the determination whether the audio segment includes the snoring sound.

In yet another embodiment, a non-transitory computer readable medium embodying a computer program is provided. The computer program includes program code that, when executed by a processor of an electronic device, causes the electronic device to process, based on a current step size of an audio stream segmenter, audio from an ambient environment of the electronic device received from a microphone, into an audio segment. The program code, when executed by the processor of the electronic device, also causes the electronic device to determine whether the audio segment includes a snoring sound, and set a next step size of the audio stream segmenter based on the determination whether the audio segment includes the snoring sound.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The term “controller” means any device, system or part thereof that controls at least one operation. Such a controller may be implemented in hardware or a combination of hardware and software and/or firmware. The functionality associated with any particular controller may be centralized or distributed, whether locally or remotely. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of this disclosure and its advantages, reference is now made to the following description, taken in conjunction with the accompanying drawings, in which:

FIG. 1 illustrates an example communication system according to embodiments of the present disclosure;

FIG. 2 illustrates an example electronic device according to embodiments of the present disclosure;

FIG. 3 illustrates an example snoring detection scenario according to embodiments of the present disclosure;

FIG. 4 illustrates an example processing pipeline for snoring sound detection according to embodiments of the present disclosure;

FIG. 5 illustrates an example method for processing an audio stream according to embodiments of the present disclosure;

FIG. 6 illustrates an example method for a quick check of audio energy according to embodiments of the present disclosure;

FIG. 7 illustrates an example method for fine classification by a snoring sound recognizer (SSR) according to embodiments of the present disclosure;

FIG. 8 illustrates another example method for processing an audio stream according to embodiments of the present disclosure;

FIG. 9 illustrates an example procedure for a periodical pattern tester (PPT) according to embodiments of the present disclosure; and

FIG. 10 illustrates an example method for ambient real-time snore detection on lightweight IoT devices with microphones according to embodiments of the present disclosure.

### DETAILED DESCRIPTION

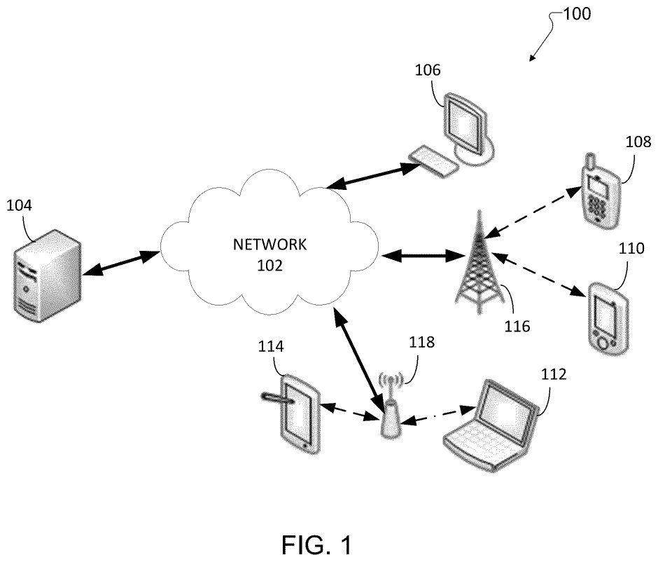

FIGS. 1 through 10, discussed below, and the various embodiments used to describe the principles of this disclosure in this patent document are by way of illustration only and should not be construed in any way to limit the scope of the disclosure. Those skilled in the art will understand that the principles of this disclosure may be implemented in any suitably arranged system or device.

Aspects, features, and advantages of the disclosure are readily apparent from the following detailed description, simply by illustrating a number of particular embodiments and implementations, including the best mode contemplated for carrying out the disclosure. The disclosure is also capable of other and different embodiments, and its several details can be modified in various obvious respects, all without departing from the spirit and scope of the disclosure. Accordingly, the drawings and description are to be regarded as illustrative in nature, and not as restrictive. The disclosure is illustrated by way of example, and not by way of limitation, in the figures of the accompanying drawings.

The present disclosure covers several components which can be used in conjunction or in combination with one another or can operate as standalone schemes. Certain embodiments of the disclosure may be derived by utilizing a combination of several of the embodiments listed below. Also, it should be noted that further embodiments may be derived by utilizing a particular subset of operational steps as disclosed in each of these embodiments. This disclosure should be understood to cover all such embodiments.

FIG. 1 illustrates an example communication system 100 according to embodiments of the present disclosure. The embodiment of the communication system 100 shown in FIG. 1 is for illustration only. Other embodiments of the communication system 100 can be used without departing from the scope of this disclosure.

The communication system 100 includes a network 102 that facilitates communication between various components in the communication system 100. For example, the network 102 can communicate IP packets, frame relay frames, Asynchronous Transfer Mode (ATM) cells, or other information between network addresses. The network 102 includes one or more local area networks (LANs), metropolitan area networks (MANs), wide area networks (WANs), all or a portion of a global network such as the Internet, or any other communication system or systems at one or more locations.

In this example, the network 102 facilitates communications between a server 104 and various client devices 106-114. The client devices 106-114 may be, for example, a smartphone (such as a UE), a tablet computer, a laptop, a personal computer, a wearable device, a head mounted display, or the like. The server 104 can represent one or more servers. Each server 104 includes any suitable computing or processing device that can provide computing services for one or more client devices, such as the client devices 106-114. Each server 104 could, for example, include one or more processing devices, one or more memories storing instructions and data, and one or more network interfaces facilitating communication over the network 102.

Each of the client devices 106-114 represent any suitable computing or processing device that interacts with at least one server (such as the server 104) or other computing device(s) over the network 102. The client devices 106-114 include a desktop computer 106, a mobile telephone or mobile device 108 (such as a smartphone), a PDA 110, a laptop computer 112, and a tablet computer 114. However, any other or additional client devices could be used in the communication system 100, such as wearable devices. Smartphones represent a class of mobile devices 108 that are handheld devices with mobile operating systems and integrated mobile broadband cellular network connections for voice, short message service (SMS), and Internet data communications. In certain embodiments, any of the client devices 106-114 can perform processes for ambient real-time snore detection.

In this example, some client devices 108-114 communicate indirectly with the network 102. For example, the mobile device 108 and PDA 110 communicate via one or more base stations 116, such as cellular base stations or eNodeBs (eNBs) or gNodeBs (gNBs). Also, the laptop computer 112 and the tablet computer 114 communicate via one or more wireless access points 118, such as IEEE 802.11 wireless access points. Note that these are for illustration only and that each of the client devices 106-114 could communicate directly with the network 102 or indirectly with the network 102 via any suitable intermediate device(s) or network(s). In certain embodiments, any of the client devices 106-114 transmit information securely and efficiently to another device, such as, for example, the server 104.

As described in more detail below, one or more of the network 102, server 104, and client devices 106-114 include circuitry, programing, or a combination thereof, to support methods for ambient real-time snore detection.

Although FIG. 1 illustrates one example of a communication system 100, various changes can be made to FIG. 1. For example, the communication system 100 could include any number of each component in any suitable arrangement. In general, computing and communication systems come in a wide variety of configurations, and FIG. 1 does not limit the scope of this disclosure to any particular configuration. While FIG. 1 illustrates one operational environment in which various features disclosed in this patent document can be used, these features could be used in any other suitable system.

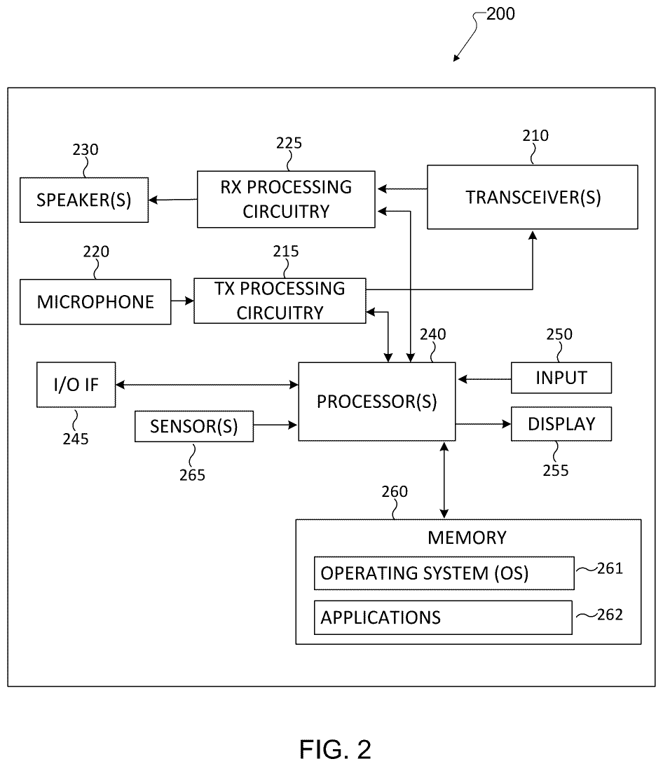

FIG. 2 illustrates an example electronic device 200 according to embodiments of the present disclosure. The electronic device 200 could represent the server 104 or one or more of the client devices 106-114 in FIG. 1. The electronic device 200 can be a mobile communication device, such as, for example, a UE, a mobile station, a subscriber station, a wireless terminal, a desktop computer (similar to the desktop computer 106 of FIG. 1), a portable electronic device (similar to the mobile device 108, the PDA 110, the laptop computer 112, or the tablet computer 114 of FIG. 1), a robot, and the like.

As shown in FIG. 2, the electronic device 200 includes transceiver(s) 210, transmit (TX) processing circuitry 215, a microphone 220, and receive (RX) processing circuitry 225. The transceiver(s) 210 can include, for example, a RF transceiver, a BLUETOOTH transceiver, a WiFi transceiver, a ZIGBEE transceiver, an infrared transceiver, and various other wireless communication signals. The electronic device 200 also includes a speaker 230, a processor 240, an input/output (I/O) interface (IF) 245, an input 250, a display 255, a memory 260, and a sensor 265. The memory 260 includes an operating system (OS) 261, and one or more applications 262.

The transceiver(s) 210 can include an antenna array including numerous antennas. For example, the transceiver(s) 210 can be equipped with multiple antenna elements. There can also be one or more antenna modules fitted on the terminal where each module can have one or more antenna elements. The antennas of the antenna array can include a radiating element composed of a conductive material or a conductive pattern formed in or on a substrate. The transceiver(s) 210 transmit and receive a signal or power to or from the electronic device 200. The transceiver(s) 210 receives an incoming signal transmitted from an access point (such as a base station, WiFi router, or BLUETOOTH device) or other device of the network 102 (such as a WiFi, BLUETOOTH, cellular, 5G, LTE, LTE-A, WiMAX, or any other type of wireless network). The transceiver(s) 210 down-converts the incoming RF signal to generate an intermediate frequency or baseband signal. The intermediate frequency or baseband signal is sent to the RX processing circuitry 225 that generates a processed baseband signal by filtering, decoding, and/or digitizing the baseband or intermediate frequency signal. The RX processing circuitry 225 transmits the processed baseband signal to the speaker 230 (such as for voice data) or to the processor 240 for further processing (such as for web browsing data).

The TX processing circuitry 215 receives analog or digital voice data from the microphone 220 or other outgoing baseband data from the processor 240. The outgoing baseband data can include web data, e-mail, or interactive video game data. The TX processing circuitry 215 encodes, multiplexes, and/or digitizes the outgoing baseband data to generate a processed baseband or intermediate frequency signal. The transceiver(s) 210 receives the outgoing processed baseband or intermediate frequency signal from the TX processing circuitry 215 and up-converts the baseband or intermediate frequency signal to a signal that is transmitted.

The processor 240 can include one or more processors or other processing devices. The processor 240 can execute instructions that are stored in the memory 260, such as the OS 261 in order to control the overall operation of the electronic device 200. For example, the processor 240 could control the reception of forward channel signals and the transmission of reverse channel signals by the transceiver(s) 210, the RX processing circuitry 225, and the TX processing circuitry 215 in accordance with well-known principles. The processor 240 can include any suitable number(s) and type(s) of processors or other devices in any suitable arrangement. For example, in certain embodiments, the processor 240 includes at least one microprocessor or microcontroller. Example types of processor 240 include microprocessors, microcontrollers, digital signal processors, field programmable gate arrays, application specific integrated circuits, and discrete circuitry. In certain embodiments, the processor 240 can include a neural network.

The processor 240 is also capable of executing other processes and programs resident in the memory 260, such as operations that receive and store data, and for example, processes that support methods for ambient real-time snore detection. The processor 240 can move data into or out of the memory 260 as required by an executing process. In certain embodiments, the processor 240 is configured to execute the one or more applications 262 based on the OS 261 or in response to signals received from external source(s) or an operator. For example, applications 262 can include a multimedia player (such as a music player or a video player), a phone calling application, a virtual personal assistant, and the like.

The processor 240 is also coupled to the I/O interface 245 that provides the electronic device 200 with the ability to connect to other devices, such as client devices 106-114. The I/O interface 245 is the communication path between these accessories and the processor 240.

The processor 240 is also coupled to the input 250 and the display 255. The operator of the electronic device 200 can use the input 250 to enter data or inputs into the electronic device 200. The input 250 can be a keyboard, touchscreen, mouse, track ball, voice input, or other device capable of acting as a user interface to allow a user to interact with the electronic device 200. For example, the input 250 can include voice recognition processing, thereby allowing a user to input a voice command. In another example, the input 250 can include a touch panel, a (digital) pen sensor, a key, or an ultrasonic input device. The touch panel can recognize, for example, a touch input in at least one scheme, such as a capacitive scheme, a pressure sensitive scheme, an infrared scheme, or an ultrasonic scheme. The input 250 can be associated with the sensor(s) 265, a camera, and the like, which provide additional inputs to the processor 240. The input 250 can also include a control circuit. In the capacitive scheme, the input 250 can recognize touch or proximity.

The display 255 can be a liquid crystal display (LCD), light-emitting diode (LED) display, organic LED (OLED), active matrix OLED (AMOLED), or other display capable of rendering text and/or graphics, such as from websites, videos, games, images, and the like. The display 255 can be a singular display screen or multiple display screens capable of creating a stereoscopic display. In certain embodiments, the display 255 is a heads-up display (HUD).

The memory 260 is coupled to the processor 240. Part of the memory 260 could include a RAM, and another part of the memory 260 could include a Flash memory or other ROM. The memory 260 can include persistent storage (not shown) that represents any structure(s) capable of storing and facilitating retrieval of information (such as data, program code, and/or other suitable information). The memory 260 can contain one or more components or devices supporting longer-term storage of data, such as a read only memory, hard drive, Flash memory, or optical disc.

The electronic device 200 further includes one or more sensors 265 that can meter a physical quantity or detect an activation state of the electronic device 200 and convert metered or detected information into an electrical signal. For example, the sensor 265 can include one or more buttons for touch input, a camera, a gesture sensor, optical sensors, cameras, one or more inertial measurement units (IMUs), such as a gyroscope or gyro sensor, and an accelerometer. The sensor 265 can also include an air pressure sensor, a magnetic sensor or magnetometer, a grip sensor, a proximity sensor, an ambient light sensor, a bio-physical sensor, a temperature/humidity sensor, an illumination sensor, an Ultraviolet (UV) sensor, an Electromyography (EMG) sensor, an Electroencephalogram (EEG) sensor, an Electrocardiogram (ECG) sensor, an IR sensor, an ultrasound sensor, an iris sensor, a fingerprint sensor, a color sensor (such as a Red Green Blue (RGB) sensor), and the like. The sensor 265 can further include control circuits for controlling any of the sensors included therein. Any of these sensor(s) 265 may be located within the electronic device 200 or within a secondary device operably connected to the electronic device 200.

Although FIG. 2 illustrates one example of electronic device 200, various changes can be made to FIG. 2. For example, various components in FIG. 2 can be combined, further subdivided, or omitted and additional components can be added according to particular needs. As a particular example, the processor 240 can be divided into multiple processors, such as one or more central processing units (CPUs), one or more graphics processing units (GPUs), one or more neural networks, and the like. Also, while FIG. 2 illustrates the electronic device 200 configured as a mobile telephone, tablet, or smartphone, the electronic device 200 can be configured to operate as other types of mobile or stationary devices.

As discussed above, some methods for snoring detection are often expensive. Furthermore, snoring detection methods such as polysomnography (PSG) are accurate, but require attaching multiple sensors to the body, usually placing airflow sensors inside the patients' noses or mouths, which often causes discomfort to patients. As an alternative to PSG, microphones can be utilized to detect snoring. Because microphones are widely available in many common electronic devices such as smartphones, laptops, and televisions, as well as Internet of Things (IoT) devices such as smart speakers, smartwatches, and the like, the ability to detect a snoring sound by using these devices may provide an affordable way of detecting snoring. This also avoids the need to place multiple sensors on the body for snoring detection, which may be impractical.

While contactless snoring detection applications are available for smartphones, these applications are best operated in a quiet environment with little background noise. Snoring detection in a noisy environment is challenging, as background noise is difficult to separate from snoring sounds. The present disclosure provides various embodiments of apparatuses and associated methods for detecting snoring sounds in the presence of background noise.

Ensuring real-time processing of audio data for snore detection while maintaining low energy consumption is another significant challenge. While advantageous for their connectivity and convenience IoT devices often lack the computational power to handle large, complex models typically designed for GPU-based systems. Various embodiments of the present disclosure provide efficient processing of audio data for snore detection on less capable devices, such as IoT devices.

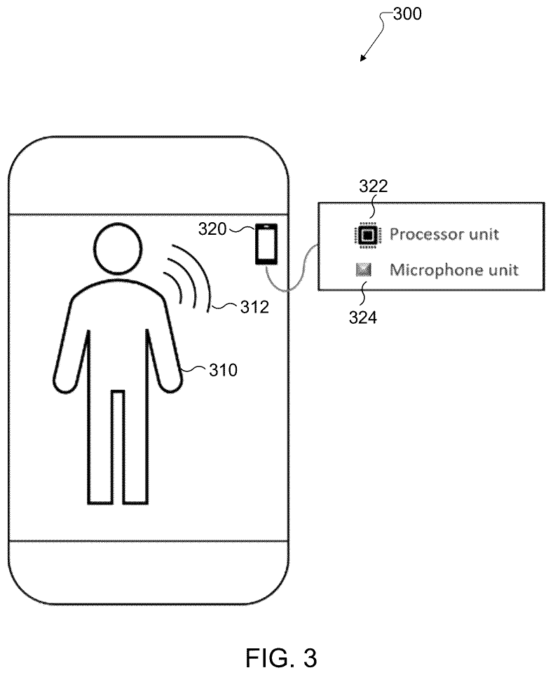

FIG. 3 illustrates an example snoring detection scenario 300 according to embodiments of the present disclosure. The embodiment of snoring detection of FIG. 3 is for illustration only. Different embodiments of snoring detection could be used without departing from the scope of this disclosure.

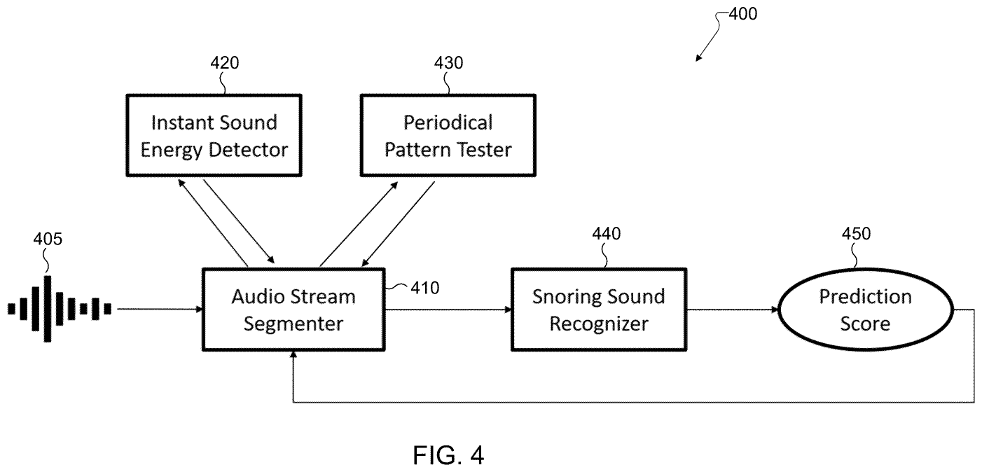

The example of FIG. 3 shows a typical snoring detection scenario where a person 310 is asleep (such as on a bed), and an IoT device 320, exemplified here by a smartphone, is strategically placed to capture snoring sounds 312. IoT device 320 includes at least one processor 322 and a microphone 324. In some embodiments, microphone 324 may be a built-in omnidirectional microphone. Unlike devices that require attachment to the body or specific orientation towards the user, IoT device 320 offers flexibility in placement. For example, proximity to the user is unnecessary, though proximity to the user may provide for improved snoring sound capture and enhanced signal-to-noise ratio (SNR). In some embodiments, the at least one processor 322 handles audio stream processing and model inference, similar as described regarding FIG. 4. In some embodiments, all data and computations related to snoring detection occur on IoT device 320 without cloud uploads. In this manner, user data privacy may be maintained. In some embodiments, detected snoring events, along with timestamps, are recorded on IoT device 320 for subsequent health analysis.

Although FIG. 3 illustrates an example snoring detection scenario 300, various changes may be made to FIG. 3. For example, various changes to type of IoT device, the location of the person, etc. could be made according to particular needs.

FIG. 4 illustrates an example processing pipeline 400 for snoring sound detection according to embodiments of the present disclosure. The embodiment of a processing pipeline of FIG. 4 is for illustration only. Different embodiments of a processing pipeline could be used without departing from the scope of this disclosure.

In the example of FIG. 4, the snoring detection process commences with audio capture by a microphone (e.g., via microphone 324 of IoT device 320). Audio from the surrounding environment captured by the microphone is continuously collected as audio samples at a sampling rate fs samples/second. In some embodiments, the microphone is omnidirectional, which allows for the microphone to be placed at any location and position without calibration in many circumstances. In situations where the target snoring sound is weak compared to a nearby noise, the microphone may be moved closer to the target user. The captured audio samples are directed to the audio stream segmenter 410 as audio stream 405 with a uniform interval of 1/fs seconds.

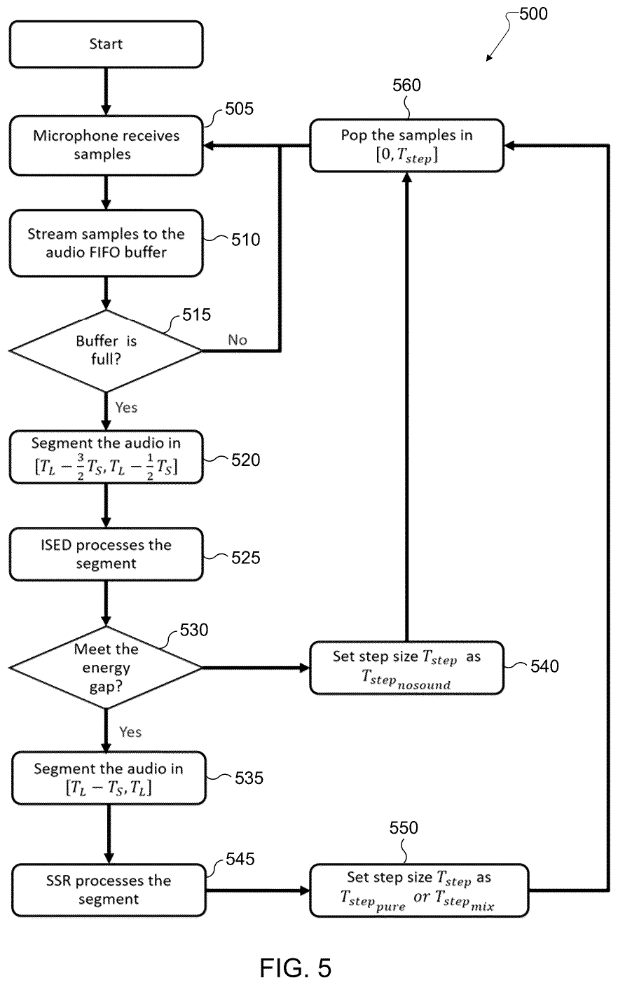

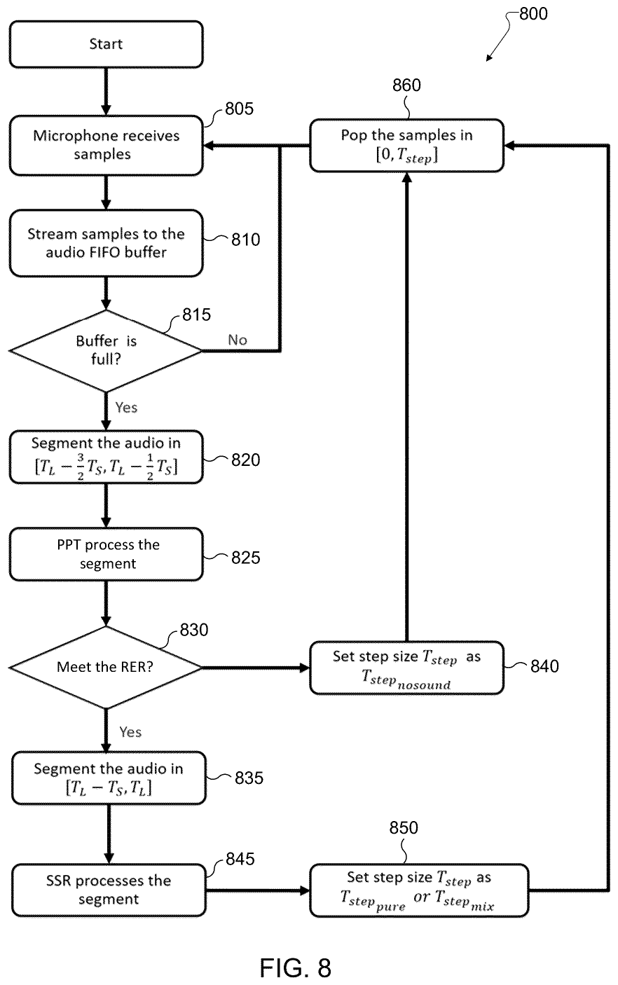

In the example of FIG. 4, audio stream segmenter 410 is utilized to intelligently segment audio stream 405 into audio clips according to system overload and detection results. audio stream segmenter 410 receives the incoming audio stream 405 and yields organized constant audio segments for the other components of processing pipeline 400. In some embodiments, audio stream segmenter 410 segments the audio stream into constant duration clips with an adaptive sliding step size, optimizing for both coverage and efficiency. In some embodiments, audio stream segmenter 410 includes an audio sample first in, first out (FIFO) buffer with a total length T_{L} and predefined parameters. The predefined parameters may include segmentation window length Tₛ, and the sliding steps Tₛₜₑₚ_{_{nosound}}, Tₛₜₑₚ_{ₚᵤᵣₑ}, Tₛₜₑₚ_{ₘᵢₓ}. In the examples of the present disclosure, the units are described in seconds, though other units may be used to define the various parameters. Some embodiments of operation of an audio stream segmenter such as audio stream segmenter 410 are further described herein with respect to FIG. 5 and FIG. 8.

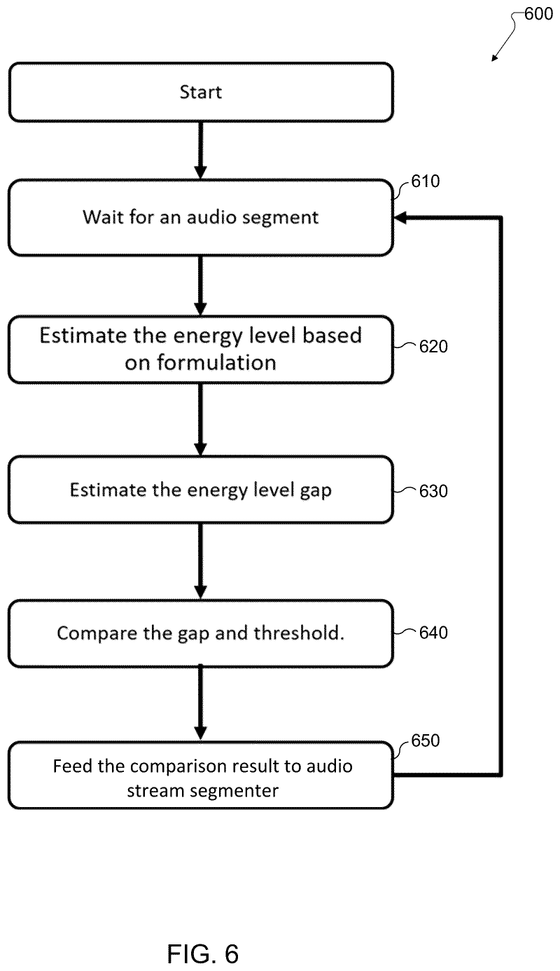

In some embodiments, each segment undergoes a preliminary evaluation by the Instant Sound Energy Detector (ISED) 420 to determine the presence of potential snoring or sound events. Some embodiments of operation of an ISED such as ISED 420 are further described herein with respect to FIG. 6. In some embodiments, segments that do not meet the criteria of ISED 420 are recycled back to audio stream segmenter 410 for capturing subsequent audio.

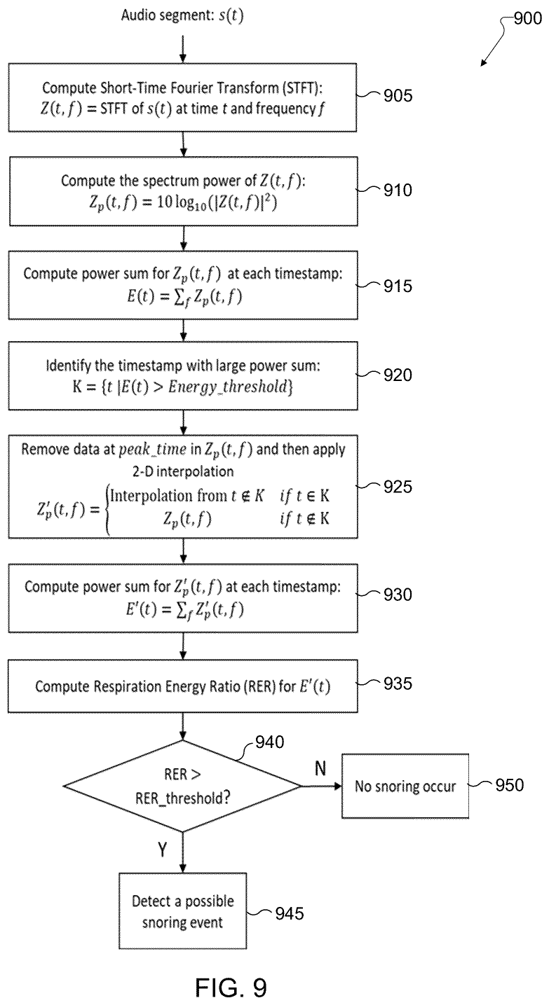

In some embodiments, Periodical Pattern Tester (PPT) 430 can check the audio pattern to determine the presence of potential snoring according to other criteria. Some embodiments of operation of a PPT such as PPT 430 are further described herein with respect to FIG. 9. In some embodiments, segments that do not meet the criteria of PPT 430 are recycled back to audio stream segmenter 410 for capturing subsequent audio.

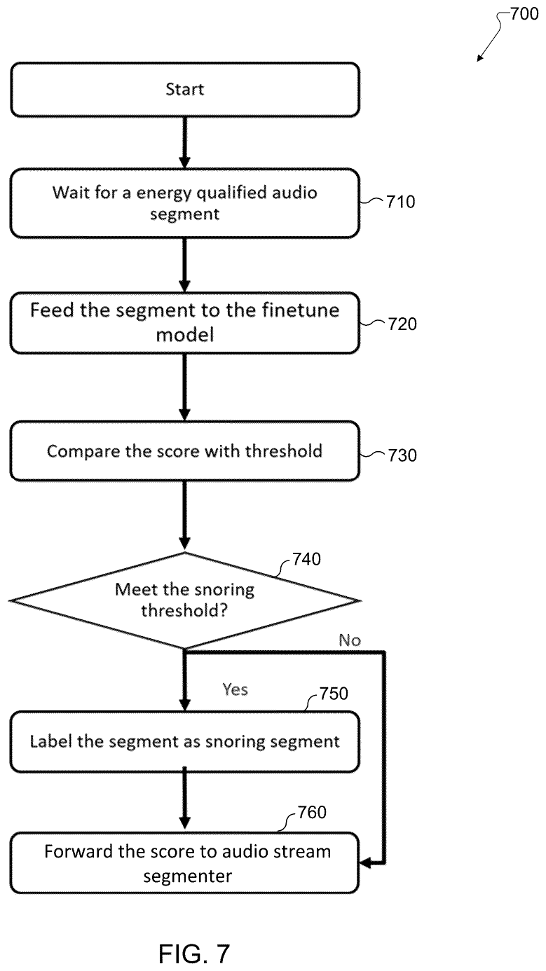

In some embodiments, qualified segments are forwarded to the Snoring Sound Recognizer (SSR) 440, which assesses the likelihood of snoring within each segment. Some embodiments of operation of an SSR such as SSR 440 are further described herein with respect to FIG. 7.

In some embodiments, SSR 440 includes a deep neural network. Many models can deal with sound classification problems and meet performance metrics on well-known benchmarks. However, such models encounter performance drops in real world scenarios due to the complex sound in a real environment and limited data samples. Snoring sound detection faces similar challenges from a real-world environment as well. For example, different people have various snoring sound patterns which vary in pitch, magnitude, and duration. It is difficult to cover these patterns efficiently. Furthermore, interference from the background can disturb the classification of a snoring sound. These sounds can mislead a model to make an incorrect classification to other sound categories. To overcome these limitations, various embodiments of SSR 440 leverage the power of a large transformer based pretrained audio model. In some embodiments, the model is pretrained on a very large-scale dataset. The pretrained model (e.g., the large transformer based pretrained audio model) can learn a general feature embedding for natural sounds. In some embodiments, to improve performance, the pretrained is finetuned with a dedicated data collection and augmentation pipeline. In some embodiments, the SSR includes both an offline finetuning stage and a real time inference stage to enable the SSR to keep high snoring detection accuracy as well as meet the latency limitations of an IoT system.

In some embodiments, for the offline finetuning stage, a large amount of clear snoring data is collected. The data includes a large number of variations of snoring sound patterns. Additionally, other types of sounds are collected. The clean snoring sound segments S and noisy environment sound segments Sₙ are mixed together by adding them together with a SNR controlled coefficient σ. This produces new augmented audio segments (noisy snoring sounds), S_{aug}, where S_{aug}=S+σSₙ. Snoring sound segments with different noise levels may be constructed by adjusting 6.

In some embodiments, a binary classification layer is attached to the pretrained model (e.g., the pretrained large model). It is finetuned with augmented audio segments. Augmented audio segments with the labels 0 and 1 are used as input. 0 refers to no snoring sound in the segments. 1 indicates that there is a snoring sound in the segments. During the training, the parameters of the pretrained model are frozen. The parameters of the binary classification layer are updated by backward propagation. This enables the finetuned layer to adapt to the specific snoring detection and keep the capability of feature extraction from the pretrained model.

In some embodiments, for the real time inference stage, the inference latency δ of the SSR is evaluated on a target device (e.g., IoT device 320 of FIG. 3). δ is related to the set up of Tₛₜₑₚ_{ₘᵢₓ} and Tₛₜₑₚ_{ₛₙₒᵣₑ}. In some cases, for proper operation, Tₛₜₑₚ_{ₘᵢₓ} and Tₛₜₑₚ_{ₛₙₒᵣₑ} should be more than δ. Then, audio stream segmenter 410 can make an appropriate sized step to avoid accumulated latency that may block the detection process.

In some embodiments, the SSR employs a scoring system to quantify the probability of snoring. In some embodiments, the SSR outputs a score in the range between 0 and 1. The score indicates the possibility of the sound to be the snoring sound. Segments exceeding a predefined threshold are classified as snoring events. A prediction score 450 is returned to the audio stream segmenter based on the assessment from the SSR.

In some embodiments the feedback from the SSR informs the audio stream segmenter for adaptive step size adjustments, enhancing the system's responsiveness and accuracy. Various embodiments of the processing pipeline 400 in FIG. 4, with various combinations of an audio stream segmenter, ISED, PPT and SSR, minimize model inference frequency while maintaining high-performance snoring detection. In some embodiments, the efficiency of processing pipeline 400 is further augmented by the fine-tuning of a large-scale pretrained model, providing robust and accurate snoring recognition across diverse scenarios.

Although FIG. 4 illustrates an example processing pipeline 400 for snoring sound detection, various changes may be made to FIG. 4. For example, some embodiments may of processing pipeline 400 may exclude ISED 420, and other embodiments may exclude PPT 530 according to particular needs.

FIG. 5 illustrates an example method 500 for processing an audio stream according to embodiments of the present disclosure. An embodiment of the method illustrated in FIG. 5 is for illustration only. One or more of the components illustrated in FIG. 5 may be implemented in specialized circuitry configured to perform the noted functions or one or more of the components may be implemented by one or more processors executing instructions to perform the noted functions. Other embodiments of a method for processing an audio stream could be used without departing from the scope of this disclosure.

In the example of FIG. 5, an incoming audio stream (e.g., audio stream 405 of FIG. 4) is processed. For example, the audio stream may be processed by an audio stream segmenter, such as audio stream segmenter 410 of FIG. 4 according to method 500.

In the example of FIG. 5, method 500 begins at step 505. At step 505, a microphone (e.g., microphone 324 of IoT device 320) captures audio from the surrounding environment similar as described regarding FIG. 4. At step 510, the audio is streamed to an audio stream segmenter (e.g., audio stream segmenter 410) similar as described regarding FIG. 4, and stored in an audio FIFO buffer. In some embodiments, the FIFO buffer may store a maximum number of samples T_{L}*fs.

At step 515, the audio stream segmenter determines whether the buffer is full. If the buffer is not full, the method returns back to step 505. Otherwise, if the buffer is full, the method proceeds at step 520. At step 520, the audio stream segmenter generates an audio segment S from the samples in the buffer. In some embodiments, the segment starts from

$${T}_{L} - \frac{3}{2} {T}_{S}$$

and ends at

$${T}_{L} - \frac{1}{2} {T}_{S} .$$

At step 525, the audio segment S is processed by an ISED (e.g., ISED 420 of FIG. 4) to determine an energy level gap within the audio segment S. For example, the ISED may process the audio segment S according to the method described regarding FIG. 6. At step 530, if the energy level gap within the audio segment S exceeds a threshold, the method proceeds at step 535. Otherwise, if the energy level gap within the audio segment S does not exceed the threshold, the method proceeds at step 540.

At step 535, the audio stream segmenter generates another audio segment from the samples in the buffer. In some embodiments, the audio segment starts from T_{L}−T_{S} and ends at T_{L}. In this manner, the audio segment is a delayed audio segment rather than being the same as the audio segment generated at step 520. The method then proceeds to step 545.

At step 540, the audio stream segmenter sets the step size Tₛₜₑₚ as Tₛₜₑₚ_{_{nosound}}. The method then proceeds to step 560.

At step 545, the audio segment generated at step 535 is processed by an SSR (e.g., SSR 440 of FIG. 4) to score the audio segment according to a metric measuring the possibility of this segment including a snoring sound. For example, the SSR may process the audio segment according to the method described regarding FIG. 7. In embodiments where the audio segment is a delayed audio segment as described regarding step 535, the audio segment may have an increased portion of a snoring sound compared to the audio segment generated in step 520. In this manner, the SSR may recognize a snoring sound withing the audio segment with a higher confidence level.

At step 550, the audio stream segmenter sets the step size Tₛₜₑₚ according to the score. If the score is greater than a threshold sₛₙₒᵣₑ or a predetermined amount below the threshold sₛₙₒᵣₑ, Tₛₜₑₚ is set as Tₛₜₑₚ_{ₚᵤᵣₑ}. Otherwise, Tₛₜₑₚ is set as Tₛₜₑₚ_{ₘᵢₓ}.

At step 560, the audio stream segmenter pops the samples in the audio FIFO buffer from the start to Tₛₜₑₚ seconds. The method then returns to step 505.

As seen above, during operation according to method 500, the audio stream segmenter receives feedback from other modules to adapt the step size to avoid unnecessary segment processing. For example, when the ISED determines there is no sound event inside the segment, the audio stream segmenter only makes a small step according to Tₛₜₑₚ_{_{nosound}}. In another example, when the SSR detects a snoring sound or a non-snoring sound with very high confidence, audio stream segmenter makes a large step according Tₛₜₑₚ_{ₛₙₒᵣₑ} to avoid overlap detection over this segment. In yet another example, when the SSR is not certain about the type of the sound, the audio stream segmenter can move a medium step according to Tₛₜₑₚ_{ₘᵢₓ} to include more effective sound in the segment and perform the SSR again. This approach may minimize the frequency to invoke the ISED and SSR while still processing all effective snoring segments.

Although FIG. 5 illustrates one example method 500 for processing an audio stream, various changes may be made to FIG. 5. For example, while shown as a series of steps, various steps in FIG. 5 could overlap, occur in parallel, occur in a different order, occur any number of times, be omitted, or replaced by other steps.

FIG. 6 illustrates an example method 600 for a quick check of audio energy according to embodiments of the present disclosure. An embodiment of the method illustrated in FIG. δ is for illustration only. One or more of the components illustrated in FIG. 6 may be implemented in specialized circuitry configured to perform the noted functions or one or more of the components may be implemented by one or more processors executing instructions to perform the noted functions. Other embodiments of a method for quick check of audio energy could be used without departing from the scope of this disclosure.

In the example of FIG. 6, method 600 begins at step 610. At step 610, an ISED (e.g., ISED 420 of FIG. 4) waits to receive an audio segment S (e.g., an audio segment generated according to step 520 in FIG. 5). In some embodiments, the audio segment S may have a length of Tₛ seconds. The length Tₛ may be set based on an observation of natural snoring sounds. For example, Tₛ may be 0.1 second.

At step 620, the energy level of the audio segment is estimated based on a formulation. For example, in some embodiments, the energy level may be estimated based on a convolution operation. In some embodiments, the formulation may be defined as follows:

$$Power = 10 {log}_{10} {S}^{2} ⊗ P\_op$$

where P_op is a power vector with the length T_{powerwin}*fs, and each element in the vector is

$$\frac{1}{{T}_{powerwin} * fs} .$$

At step 630, an energy level gap of the audio segment is estimated. In some embodiments, the energy level gap is the calculated difference between a maximal energy level and the 10-percentile energy level of the audio segment. This may avoid rare cases of outlier low energy level as opposed to calculating the difference between the maximal energy level and the minimal energy level.

At step 640, the energy level gap is compared with a threshold. In the example of FIG. 6, if the energy level gap meets the threshold, the audio segment has a high potential to have a sound event and is used for a fine classification by an SSR. Otherwise, the segment is determined as having no potential snoring sound.

At step 650, the result from step 640 is returned to the audio stream segmenter.

By utilizing method 600, the ISED performs a coarse detection. Method 600 filters out most segments without the snoring sound and only passes qualified segments to the SSR. This reduces the computation complexity significantly and provides for real time processing of the full processing pipeline 400 by an IoT device.

Although FIG. 6 illustrates one example method 600 for a quick check of audio energy, various changes may be made to FIG. 6. For example, while shown as a series of steps, various steps in FIG. 6 could overlap, occur in parallel, occur in a different order, occur any number of times, be omitted, or replaced by other steps.

FIG. 7 illustrates an example method for fine classification by an SSR 700 according to embodiments of the present disclosure. An embodiment of the method illustrated in FIG. 7 is for illustration only. One or more of the components illustrated in FIG. 7 may be implemented in specialized circuitry configured to perform the noted functions or one or more of the components may be implemented by one or more processors executing instructions to perform the noted functions. Other embodiments of a method for fine classification by an SSR could be used without departing from the scope of this disclosure.

In the example of FIG. 7, an SSR, such as SSR 440 of FIG. 4, is used to perform snoring sound classification. In some embodiments, the SSR includes a deep neural network.

In the example of FIG. 7, method 700 begins at step 710. At step 710, an SSR (e.g., SSR 440 of FIG. 4) waits to receive an audio segment S (e.g., an audio segment generated according to step 535 in FIG. 5).

At step 720, the SSR evaluates the audio segment via a finetuned snoring sound model. The evaluation generates a score (e.g., between 0 and 1).

At step 730, the SSR compares the score from step 720 against a threshold. At step 740, if the threshold is exceeded, the method proceeds to step 750. Otherwise, the method proceeds to step 760.

At step 750, the SSR labels the segment as a snoring segment. This information may be used to further finetune the model.

At step 760, the SSR forwards the score from step 720 to an audio stream segmenter (e.g., audio stream segmenter 410 of FIG. 4).

Although FIG. 7 illustrates one example method for fine classification by an SSR 700, various changes may be made to FIG. 7. For example, while shown as a series of steps, various steps in FIG. 7 could overlap, occur in parallel, occur in a different order, occur any number of times, be omitted, or replaced by other steps.

Because snoring is caused by obstructed breathing, the temporal periodicity of a snoring sound may be the same as the temporal periodicity of human respiration. This method detects whether an audio segment has the temporal periodicity that falls within the range of human respiration's periodicity. In another embodiment of this disclosure, instead of using relative energy as described regarding FIG. 5, a PPT detects whether an audio segment has a temporal periodicity that falls within the range of human respiration's periodicity to detect the potential occurrence of a snoring event as shown in FIG. 8.

FIG. 8 illustrates another example method 800 for processing an audio stream according to embodiments of the present disclosure. An embodiment of the method illustrated in FIG. 8 is for illustration only. One or more of the components illustrated in FIG. 8 may be implemented in specialized circuitry configured to perform the noted functions or one or more of the components may be implemented by one or more processors executing instructions to perform the noted functions. Other embodiments of a method for processing an audio stream could be used without departing from the scope of this disclosure.

In the example of FIG. 8, an incoming audio stream (e.g., audio stream 405 of FIG. 4) is processed. For example, the audio stream may be processed by an audio stream segmenter, such as audio stream segmenter 410 of FIG. 4 according to method 800.

In the example of FIG. 8, method 800 begins at step 805. At step 805, a microphone (e.g., microphone 324 of IoT device 320) captures audio from the surrounding environment similar as described regarding FIG. 4. At step 810, the audio is streamed to an audio stream segmenter (e.g., audio stream segmenter 410) similar as described regarding FIG. 4, and stored in an audio FIFO buffer. In some embodiments, the FIFO buffer may store a maximum number of samples T_{L}*fs.

At step 815, the audio stream segmenter determines whether the buffer is full. If the buffer is not full, the method returns back to step 805. Otherwise, if the buffer is full, the method proceeds at step 820. At step 820, the audio stream segmenter generates an audio segment S from the samples in the buffer. In some embodiments, the segment starts from

$${T}_{L} - \frac{3}{2} {T}_{S}$$

and ends at

$${T}_{L} - \frac{1}{2} {T}_{S} .$$

At step 825, the audio segment S is processed by an ISED (e.g., ISED 420 of FIG. 4) to determine a Respiration Energy Ratio (RER) within the audio segment S. For example, the ISED may process the audio segment S according to the method described regarding FIG. 9. At step 830, if the RER within the audio segment S exceeds a threshold, the method proceeds at step 835. Otherwise, if the RER within the audio segment S does not exceed the threshold, the method proceeds at step 840.

At step 835, the audio stream segmenter generates another audio segment from the samples in the buffer. In some embodiments, the audio segment starts from T_{L}−T_{S} and ends at T_{L}. In this manner, the audio segment is a delayed audio segment rather than being the same as the audio segment generated at step 820. The method then proceeds to step 845.

At step 840, the audio stream segmenter sets the step size Tₛₜₑₚ as Tₛₜₑₚ_{_{nosound}}. The method then proceeds to step 860.

At step 845, the audio segment generated at step 835 is processed by an SSR (e.g., SSR 440 of FIG. 4) to score the audio segment according to a metric measuring the possibility of this segment including a snoring sound. For example, the SSR may process the audio segment according to the method described regarding FIG. 7. In embodiments where the audio segment is a delayed audio segment as described regarding step 835, the audio segment may have an increased portion of a snoring sound compared to the audio segment generated in step 820. In this manner, the SSR may recognize a snoring sound withing the audio segment with a higher confidence level.

At step 850, the audio stream segmenter sets the step size Tₛₜₑₚ according to the score. If the score is greater than a threshold sₛₙₒᵣₑ or a predetermined amount below the threshold Sₛₙₒᵣₑ, Tₛₜₑₚ is set as Tₛₜₑₚ_{ₚᵤᵣₑ}. Otherwise, Tₛₜₑₚ is set as Tₛₜₑₚ_{ₘᵢₓ}.

At step 860, the audio stream segmenter pops the samples in the audio FIFO buffer from the start to Tₛₜₑₚ seconds. The method then returns to step 805.

As seen above, during operation according to method 800, the audio stream segmenter receives feedback from other modules to adapt the step size to avoid unnecessary segment processing. For example, when the ISED determines there is no sound event inside the segment, the audio stream segmenter only makes a small step according to Tₛₜₑₚ_{_{nosound}}. In another example, when the SSR detects a snoring sound or a non-snoring sound with very high confidence, audio stream segmenter makes a large step according Tₛₜₑₚ_{ₛₙₒᵣₑ} to avoid overlap detection over this segment. In yet another example, when the SSR is not certain about the type of the sound, the audio stream segmenter can move a medium step according to Tₛₜₑₚ_{ₘᵢₓ} to include more effective sound in the segment and perform the SSR again. This approach may minimize the frequency to invoke the ISED and SSR while still processing all effective snoring segments.

Although FIG. 8 illustrates one example method 800 for processing an audio stream, various changes may be made to FIG. 8. For example, while shown as a series of steps, various steps in FIG. 8 could overlap, occur in parallel, occur in a different order, occur any number of times, be omitted, or replaced by other steps.

FIG. 9 illustrates an example procedure for a PPT 900 according to embodiments of the present disclosure. An embodiment of the method illustrated in FIG. 9 is for illustration only. One or more of the components illustrated in FIG. 9 may be implemented in specialized circuitry configured to perform the noted functions or one or more of the components may be implemented by one or more processors executing instructions to perform the noted functions. Other embodiments of a procedure for a PPT could be used without departing from the scope of this disclosure.

In the example of FIG. 9, procedure 900 begins at step 905. At step 905, to make the procedure robust to background noises, the data that contains large background noises is detected and removed. Specifically, the spectrum power of the input audio segment, Zₚ(t,f), is computed by using the Short-Time Fourier Transform (STFT). Then, at step 910 the spectrum power sum at each timestamp, E(t), is computed to detect the large background noises, i.e., the timestamps with abnormal large E(t) at step 915 are identified at step 920 as having an occurrence of large background noises.

At step 925, the data at these timestamps is removed from Zₚ(t,f), and the removed data is replaced by the 2-D interpolation of the remaining data to get a noise-reduced spectrum power Zₚ′(t,f).

At step 930, the spectrum power sum at each timestamp, E′(t), is computed on Zₚ′(t,f) to detect the temporal periodicity.

At step 935, to detect whether the temporal periodicity falls within the range of human respiration periodicity, the PPT computes the Respiration Energy Ratio (RER) of E′(t). If RER is larger than a certain threshold at step 940, then snoring may happen in the input audio segment with high possibility. In this case, at step 945 this audio segment is input to the Snoring Sound Recognizer described in FIG. 8 for snoring classification. If the RER is less than the threshold, at step 950, the audio segment will not be input to the Snoring Sound Recognizer for snoring classification to reduce the computational burden of the system.

Although FIG. 9 illustrates one example procedure for a PPT 900, various changes may be made to FIG. 9. For example, while shown as a series of steps, various steps in FIG. 9 could overlap, occur in parallel, occur in a different order, occur any number of times, be omitted, or replaced by other steps.

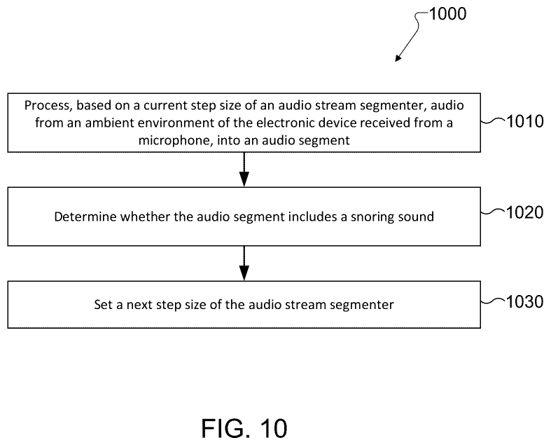

FIG. 10 illustrates an example method for ambient real-time snore detection on lightweight IoT devices with microphones 1000 according to embodiments of the present disclosure. An embodiment of the method illustrated in FIG. 10 is for illustration only. One or more of the components illustrated in FIG. 10 may be implemented in specialized circuitry configured to perform the noted functions or one or more of the components may be implemented by one or more processors executing instructions to perform the noted functions. Other embodiments of a method for ambient real-time snore detection on lightweight IoT devices with microphones could be used without departing from the scope of this disclosure.

In the example of FIG. 10, method 1000 begins at step 1010. At step 1010, an IoT device (e.g., IoT device 320 of FIG. 3) processes (e.g., via processor 322), based on a current step size of an audio stream segmenter (e.g., audio stream segmenter 410 of FIG. 4) audio from an ambient environment of the electronic device received from a microphone (e.g. microphone 322 of IoT device 324).

At step 1020, the IoT device determines whether the audio segment includes a snoring sound. In some embodiments, to determine whether the audio segment includes the snoring sound at step 1020, the IoT device determines whether the audio segment potentially includes a snoring event. Based on a determination that the audio segment potentially includes a snoring event, the IoT device determines via a finetuned snoring sound model whether the audio segment includes the snoring sound. In some embodiments, a determination that the audio segment does not potentially include a snoring event is indicative that the audio segment does not include a snoring sound. In some embodiments, when a prediction score received from the finetuned snoring sound model exceeds a threshold, the IoT devices determines that the audio segment includes the snoring sound. In some embodiments,

In some embodiments, to determine whether the audio segment potentially includes a snoring event, the IoT device determines whether an estimated energy level of the audio segment exceeds a threshold. In some embodiments, a determination that the estimated energy level of the audio segment exceeds the threshold is indicative that the audio segment potentially includes the snoring event. In some embodiments, to determine whether the estimated energy level of the audio segment exceeds the threshold, the IoT device determines a difference between a maximal energy level of the audio segment and a baseline energy level of the audio segment, and determines whether the difference exceeds the threshold. In some embodiments, a determination that the difference exceeds the threshold is indicative that the estimated energy level of the audio segment exceeds the threshold.

In some embodiments, to determine whether the audio segment potentially includes a snoring event, the IoT devices determines whether a temporal periodicity of the audio segment falls within a range of a human respiration periodicity. In some embodiments, a determination that the temporal periodicity of the audio segment falls within the range of the human respiration periodicity is indicative that the audio segment potentially includes the snoring event. In some embodiments, to determine whether the temporal periodicity of the audio segment falls within the range of the human respiration periodicity, the IoT determines a RER for at least a portion of the audio segment, and determines whether the RER exceeds an RER threshold. In some embodiments, a determination that the RER exceeds an RER threshold is indicative that the temporal periodicity of the audio segment falls within the range of the human respiration periodicity.

At step 1030, the IoT device sets a next step size of the audio stream segmenter. The next step size may be set based on the determination whether the audio segment includes the snoring sound. In some embodiments, when the audio segment does not potentially include the snoring event, the IoT device sets the next step size of the audio stream segmenter to a first step size. In some embodiments, when a prediction score exceeds a threshold, the IoT device sets the next step size of the audio stream segmenter to a second step size. In some embodiments, when the prediction score does not exceed the threshold, the IoT device sets the next step size of the audio stream segmenter to a third step size.

Although FIG. 10 illustrates one example method for ambient real-time snore detection on lightweight IoT devices with microphones 1000, various changes may be made to FIG. 10. For example, while shown as a series of steps, various steps in FIG. 10 could overlap, occur in parallel, occur in a different order, occur any number of times, be omitted, or replaced by other steps.

Any of the above variation embodiments can be utilized independently or in combination with at least one other variation embodiment. The above flowcharts illustrate example methods that can be implemented in accordance with the principles of the present disclosure and various changes could be made to the methods illustrated in the flowcharts herein. For example, while shown as a series of steps, various steps in each figure could overlap, occur in parallel, occur in a different order, or occur multiple times. In another example, steps may be omitted or replaced by other steps.

Although the present disclosure has been described with exemplary embodiments, various changes and modifications may be suggested to one skilled in the art. It is intended that the present disclosure encompass such changes and modifications as fall within the scope of the appended claims. None of the description in this application should be read as implying that any particular element, step, or function is an essential element that must be included in the claim scope. The scope of patented subject matter is defined by the claims.

What is claimed is:

1. An electronic device comprising:
a processor; and
a microphone operatively coupled to the processor, the microphone configured to send audio, from an ambient environment of the electronic device, to the processor,
wherein, the processor is configured to:
process, based on a current step size of an audio stream segmenter, the audio received from the microphone into an audio segment;
determine whether the audio segment includes a snoring sound; and
set a next step size of the audio stream segmenter based on the determination whether the audio segment includes the snoring sound.

2. The electronic device of claim 1, wherein:
to determine whether the audio segment includes the snoring sound, the processor is further configured to:
determine whether the audio segment potentially includes a snoring event;
based on a determination that the audio segment potentially includes a snoring event, determine via finetuned snoring sound model whether the audio segment includes the snoring sound; and
when the audio segment does not potentially include the snoring event, set the next step size of the audio stream segmenter to a first step size; and
a determination that the audio segment does not potentially include a snoring event is indicative that the audio segment does not include a snoring sound.

3. The electronic device of claim 2, wherein the processor is further configured to:
when a prediction score received from the finetuned snoring sound model exceeds a threshold, determine that the audio segment includes the snoring sound;
when the prediction score exceeds the threshold, set the next step size of the audio stream segmenter to a second step size; and
when the prediction score does not exceed the threshold, set the next step size of the audio stream segmenter to a third step size.

4. The electronic device of claim 2, wherein:
to determine whether the audio segment potentially includes a snoring event, the processor is further configured to determine whether an estimated energy level of the audio segment exceeds a threshold; and
a determination that the estimated energy level of the audio segment exceeds the threshold is indicative that the audio segment potentially includes the snoring event.

5. The electronic device of claim 4, wherein:
to determine whether the estimated energy level of the audio segment exceeds the threshold the processor is further configured to:
determine a difference between a maximal energy level of the audio segment and a baseline energy level of the audio segment; and
determine whether the difference exceeds the threshold; and
a determination that the difference exceeds the threshold is indicative that the estimated energy level of the audio segment exceeds the threshold.

6. The electronic device of claim 2, wherein:
to determine whether the audio segment potentially includes a snoring event, the processor is further configured to determine whether a temporal periodicity of the audio segment falls within a range of a human respiration periodicity; and
a determination that the temporal periodicity of the audio segment falls within the range of the human respiration periodicity is indicative that the audio segment potentially includes the snoring event.

7. The electronic device of claim 6, wherein:
to determine whether the temporal periodicity of the audio segment falls within the range of the human respiration periodicity, the processor is further configured to:
determine a respiration energy ratio (RER) for at least a portion of the audio segment; and
determine whether the RER exceeds an RER threshold; and
a determination that the RER exceeds an RER threshold is indicative that the temporal periodicity of the audio segment falls within the range of the human respiration periodicity.

8. A method of operating an electronic device, the method comprising:
processing, based on a current step size of an audio stream segmenter, audio from an ambient environment of the electronic device received from a microphone, into an audio segment;
determining whether the audio segment includes a snoring sound; and
setting a next step size of the audio stream segmenter based on the determination whether the audio segment includes the snoring sound.

9. The method of claim 8, wherein to determine whether the audio segment includes the snoring sound, the method further includes:
determining whether the audio segment potentially includes a snoring event;
based on a determination that the audio segment potentially includes a snoring event, determining via a finetuned snoring sound model whether the audio segment includes the snoring sound; and
when the audio segment does not potentially include the snoring event, setting the next step size of the audio stream segmenter to a first step size,
wherein a determination that the audio segment does not potentially include a snoring event is indicative that the audio segment does not include a snoring sound.

10. The method of claim 9, further comprising:
when a prediction score received from the finetuned snoring sound model exceeds a threshold, determining that the audio segment includes the snoring sound;
when the prediction score exceeds the threshold, setting the next step size of the audio stream segmenter to a second step size; and
when the prediction score does not exceed the threshold, setting the next step size of the audio stream segmenter to a third step size.

11. The method of claim 9, wherein:
to determine whether the audio segment potentially includes a snoring event, the method further comprises determining whether an estimated energy level of the audio segment exceeds a threshold; and
a determination that the estimated energy level of the audio segment exceeds the threshold is indicative that the audio segment potentially includes the snoring event.

12. The method of claim 11, wherein:
to determine whether the estimated energy level of the audio segment exceeds the threshold the method further comprises:
determining a difference between a maximal energy level of the audio segment and a baseline energy level of the audio segment; and
determining whether the difference exceeds the threshold; and
a determination that the difference exceeds the threshold is indicative that the estimated energy level of the audio segment exceeds the threshold.

13. The method of claim 9, wherein:
to determine whether the audio segment potentially includes a snoring event, the method further comprises determining whether a temporal periodicity of the audio segment falls within a range of a human respiration periodicity; and
a determination that the temporal periodicity of the audio segment falls within the range of the human respiration periodicity is indicative that the audio segment potentially includes the snoring event.

14. The method of claim 13, wherein:
to determine whether the temporal periodicity of the audio segment falls within the range of the human respiration periodicity, the method further comprises:
determining a respiration energy ratio (RER) for at least a portion of the audio segment; and
determining whether the RER exceeds an RER threshold; and
a determination that the RER exceeds an RER threshold is indicative that the temporal periodicity of the audio segment falls within the range of the human respiration periodicity.

15. A non-transitory computer readable medium embodying a computer program, the computer program comprising program code that, when executed by a processor of an electronic device, causes the electronic device to:
process, based on a current step size of an audio stream segmenter, audio from an ambient environment of the electronic device received from a microphone, into an audio segment;
determine whether the audio segment includes a snoring sound; and
set a next step size of the audio stream segmenter based on the determination whether the audio segment includes the snoring sound.

16. The non-transitory computer readable medium of claim 15, wherein to determine whether the audio segment includes the snoring sound, the program code, when executed by the processor of the electronic device, further causes the electronic device to:
determine whether the audio segment potentially includes a snoring event;
based on a determination that the audio segment potentially includes a snoring event, determine via a finetuned snoring sound model whether the audio segment includes the snoring sound; and
when the audio segment does not potentially include the snoring event, set the next step size of the audio stream segmenter to a first step size,
wherein a determination that the audio segment does not potentially include a snoring event is indicative that the audio segment does not include a snoring sound.

17. The non-transitory computer readable medium of claim 16, further wherein the program code, when executed by the processor of the electronic device, further causes the electronic device to:
when a prediction score received from the finetuned snoring sound model exceeds a threshold, determine that the audio segment includes the snoring sound;
when the prediction score exceeds the threshold, set the next step size of the audio stream segmenter to a second step size; and
when the prediction score does not exceed the threshold, set the next step size of the audio stream segmenter to a third step size.

18. The non-transitory computer readable medium of claim 16, wherein:
to determine whether the audio segment potentially includes a snoring event, the program code, when executed by the processor of the electronic device, further causes the electronic device to determine whether an estimated energy level of the audio segment exceeds a threshold; and
a determination that the estimated energy level of the audio segment exceeds the threshold is indicative that the audio segment potentially includes the snoring event.

19. The non-transitory computer readable medium of claim 18, wherein:
to determine whether the estimated energy level of the audio segment exceeds the threshold the program code, when executed by the processor of the electronic device, further causes the electronic device to:
determine a difference between a maximal energy level of the audio segment and a baseline energy level of the audio segment; and
determine whether the difference exceeds the threshold; and
a determination that the difference exceeds the threshold is indicative that the estimated energy level of the audio segment exceeds the threshold.

20. The non-transitory computer readable medium of claim 16, wherein:
to determine whether the audio segment potentially includes a snoring event, the program code, when executed by the processor of the electronic device, further causes the electronic device to determine whether a temporal periodicity of the audio segment falls within a range of a human respiration periodicity; and
a determination that the temporal periodicity of the audio segment falls within the range of the human respiration periodicity is indicative that the audio segment potentially includes the snoring event.
